(19) United States
(12) Patent Application Publication
(10) Pub. No.: US 2011/0197289 A1
(43) Pub. Date: Aug. 11, 2011

(54) Storage of Entomopathogenic Nematodes

(76) Inventors: Shahina Fayyaz, Karachi (PK); Tabassum Ara Khanum, Karachi (PK); Manzoor Hussain Soomro, Iscamabad (PK)

(21) Appl. No.: 12/702,053

(22) Filed: Feb. 8, 2010

(51) Int. Cl. A01K 67/033

(52) U.S. Cl. 800/8

(57) ABSTRACT

This invention relates to methods of prolonged storage of the species of entomopathogenic nematodes belonging to the genera of Steinernema and Heterorhabditis for use as bio-pesticides wherein storage in surfactant solution or antimicrobial solution on sterilized polyurethane foam allows maintaining infectivity of at least 50% for six months at 10-15 C storage conditions.

### BACKGROUND

For the control of important insect pests in agricultural crops a wide range of chemical pesticides have been indiscriminately used for many decades. The long-lasting impact of the chemicals on the non-target organisms, development of insect resistance to chemical pesticides and their hazardous effects on the human and the environment stimulated the interest of the scientists for an alternate control measure through the bio-control means for managing the insect pests as to increase the agricultural productivity. Among the insect pathogens, entomopathogenic nematodes have been considered as exceptionally potential and effective bio-control agents having non-polluting properties, for the control of major insect pests, which cause serious damage to food and fiber crops of agricultural importance.

Management by eco-friendly methods using entomopathogenic nematodes as bio-control agents has gained momentum in the recent past. Use of entomopathogenic nematodes for the management of insect pests is easy in application, free from environmental pollution and improves soil health, structurally and nutritionally. As entomopathogenic nematodes of the genera Heterorhabditis and Steinernema have a wide distribution and occur in a variety of soil types and habitats, these can be used for insect control as an alternative to chemical insecticides in agriculture. For this purpose mass in vitro rearing of these nematodes on solid culture has been reported.

The storage of entomopathogenic nematodes (EPN) for control of insect pests has also been reported earlier: Yukawa et al., 1988 (U.S. Pat. No. 4,765,275) reported the methods for storage for transport of nematodes. In this method a cream of infective juveniles mixed with an absorbent and stored under anaerobic conditions for long period of time at low temperature. In 1991 Bedding (U.S. Pat. No. 5,042,427) described storage of infective stage juveniles of entomopathogenic nematodes in homogenous mixture of cream and clay. In 1994 Bedding (WO 1994005150) described the method of storage of third stage infective juveniles of EPN of the genera Steinernema and Heterorhabditis using polyacrylamide gel. Tachibana et al., 1998 (PCT/JP9603067) described, mixture of nematode clay and water preserved at low temperature for several months.

The present invention relates to the prolonged storage of entomopathogenic nematodes. Entomopathogenic nematodes have several important attributes that make them excellent candidates for biological control of insect pests. They are specialized to carry and introduce symbiotic bacteria into the insect hemocoel, have a broad host range that includes the majority of insect orders and families. Several species can be cultured artificially on a large scale, which makes it possible to commercially produce large quantities. They have a limited impact on non-target organisms and are not disruptive to the environment. Numerous insect pests of different crops are controlled by parasitic nematodes. Insect hosts include several species of root weevils and flea beetles, mint root borer and other species of stem borers, white grubs, and caterpillars. Parasitic nematodes have been used successfully to control insect pests on mint, citrus, cranberry, small fruits, lawn, ornamentals and vegetable crops. Among the insects, pathogens, entomopathogenic nematodes have been considered as exceptionally effective bio-control agents having non-polluting properties.

The most important families of the nematodes affecting insects include Steinernematidae Chitwood & Chitwood, 1937 and Heterorhabditidae Poinar, 1976. The genera used for biological pesticides are Steinernema (Travassos, 1927) and Heterorhabditis (Poinar, 1976). Several species used in the current study were reported by Shahina & Maqbool (1996) including S. pakistanese Shahina et al., 2001 and S. asiaticum Anis et al., 2002, S. feltiae Filipjev, 1934, Steinernema abbasi Elawad et al., 1997 and Steinernema siamkayai Stock et al., 1998 and two species of the genus Heterorhabditis, H. indica Poinar et al., 1992 and H. bacteriophora Poinar, 1976.

In this invention, the above mentioned seven virulent nematode species of the genera Steinernema and Heterorhabditis were cultured on mass scale using the chicken offal method (Bedding, 1984). The cultures were stored at 10-15° C. in distilled water with a drop of triton x-100 per 100 ml culture. This technique of mass culturing of infective juveniles (IJs) was used for the first time in Pakistan (Tabassum & Shahina, 2004). These EPNs have a mutualistic association with entomopathogenic bacteria (EPB) belonging to the genera Xenorhabdus (Thomus & Poinar, 1979) and Photorhabdus (Boemare et al., 1993). Three different species of bacteria Photorhabdus luminescence (Thomus & Poinar, 1979), Xenorhabdus nematophila (Akhurst & Boemare, 1988) and X. bovienii (Akhurst & Boemare, 1988) have been reported for the first time from Pakistan (Shahina et al., 2004).

### SUMMARY OF INVENTION

The entomopathogenic nematode viz., S. pakistanese Shahina et al., 2001; S. asiaticum Anis et al., 2002; S. feltiae Filipjev, 1934; Steinernema abbasi Elawad et al., 1997; Steinernema siamkayai Stock et al., 1998; Heterorhabditis indica Poinar et al., 1992 and H. bacteriophora Poinar, 1976 were extracted from soil samples by using Galleria baiting technique (Bedding & Akhurst, 1975). The collected nematode populations were maintained on Galleria mellonella larvae in the laboratory. The adults were obtained by dissecting infected G. mellonella larvae periodically in Ringer solution. Infective Juveniles (IJs) were collected by the White trap method (White, 1927). Nematodes were killed by applying gradual heat, fixed and processed in anhydrous glycerine using the method described by Poinar (1975). Measurements of all specimens were taken by an ocular micrometer. Identification of nematodes was made using morphometric characteristics as given by Nguyen & Smart (1996).

Entomopathogenic nematodes viz., S. pakistanese; S. asiaticum; S. feltiae; Steinernema abbasi; Steinernema siamkayai; Heterorhabditis indica and H. bacteriophora were reared in monoxenic culture on chicken offal medium described by Bedding (1984). The third infective stage juvenile collects from medium, washed with tap water and were kept in distilled water with a drop of Triton X-100 (a wetting agent that prevents nematodes from striking to the side of the container) or 0.1% formalin and stored on moist autoclaved polyether polyurethane foam. The foam was coated with IJs more than 10 times of foam weight placed in sterilized container and stored in growth chamber at 10-15° C.

The effect of prolonged storage on infectivity of EPN isolates viz., S. pakistanese; S. asiaticum; S. feltiae; Steinernema abbasi; Steinernema siamkayai; Heterorhabditis indica and H. bacteriophora at 10-15° C. was assessed. Infectivity was tested by using the Galleria Sand Bioassay wherein 25 G. mellonella larvae were placed in a 9 cm Petri dish then covered with the layer of moist sterile sand. Moist sand was obtained by mixing water in the ratio of 10-8% w/w with dried sterile soil. Infective juveniles suspended in 200 μl of sterilized distilled water and distributed evenly on the top of the sand. Six different age groups of infective stage juveniles viz., freshly hatched, 1-12 month old IJs were used. 500 IJs were inoculated to check the mortality of G. mellonella or infectivity of different age groups of infective stage juveniles. For each treatment three replicates were used.

The result of the experiments reported here of the infectivity of IJs stored at 15-20° C. for various periods from time of emergence from the host cadaver are as follows. The maximum infectivity of EPN isolates viz., S. pakistanense, S. asiaticum, S. feltiae, S. abbasi, S. siamkayai, H. bacteriophora and H. indica was observed in freshly hatched infective stage juveniles. The minimum infectivity was recorded in 8-12 month old storage of 3^{rd} stage infective juveniles in above mentioned isolates. Twelve month old infective juveniles lost the infectivity to kill the host.

### DETAILED DESCRIPTION OF INVENTION

The monoxenic solid artificial medium (chicken offal) proves useful for large-scale production of these nematodes and is being used globally. The techniques described by Bedding (1984) for the production of nematodes IJs per unit time were followed. With Bedding's method, normally 5-7 million infective juveniles of Steinernema were produced in a single 500 ml flask containing 80 g of chicken offal medium and stored in flasks containing distilled water at 10-15° C. for 1-12 months. IJs as the infective stage juveniles reared in above mentioned media, kept their infectivity for up-to 8 months. The infective juveniles of Steinernematids and Heterorhabditids were stored on moist autoclaved polyether polyurethane foam. The foam was coated with IJs more than 10 times of foam placed in sterilized container and stored in growth chamber at 10-15° C.

The infectivity of the above mentioned seven species during prolonged storage was analyzed by a three-way ANOVA. The analysis of variance indicated that there were significant differences between infectivity of fresh to 12 months storage IJs (F=23.2; df=7; P=0.05) but among species, there were no significant differences (F=0.05; df=6; P=0.05) that means all seven nematode species were highly infective during fresh to eight months storage.

Seven entomopathogenic nematode isolates viz., S. pakistanese Shahina et al., 2001; S. asiaticum Anis et al., 2002; S. feltiae Filipjev, 1934; Steinernema abbasi Elawad et al., 1997; Steinernema siamkayai Stock et al., 1998; Heterorhabditis indica Poinar et al., 1992 and H. bacteriophora Poinar, 1976 were reared in monoxenic culture on chicken offal medium described by Bedding (1984). The third infective stage juvenile collects from medium, washed with tap water and were kept in distilled water with a drop of Triton X-100 (a wetting agent that prevents nematodes from striking to the side of the container) or 0.1% formalin and stored on moist autoclaved polyether polyurethane foam. The foam was coated with IJs more than 10 times of foam weight, placed in sterilized container and stored in growth chamber.

The effect of prolonged storage on infectivity of EPN isolates S. pakistanese Shahina et al., 2001; S. asiaticum Anis et al., 2002; S. feltiae Filipjev, 1934; Steinernema abbasi Elawad et al., 1997; Steinernema siamkayai Stock et al., 1998; Heterorhabditis indica Poinar et al., 1992 and H. bacteriophora Poinar, 1976 at 10-15° C. were tested by using the Galleria Sand Bioassay; wherein, 25 G. mellonella larvae were placed in a 9 cm Petri dish then covered with a layer of moist sterile sand. Moist sand was obtained by mixing water in the ratio of 10-18% w/w with dried sterile soil. Infective juveniles were suspended in 200 μl of sterilized distilled water and distributed evenly on the top of the sand. Six different age groups of infective stage juveniles viz., freshly hatched, 1-12 month old IJs were used. 500 IJs were inoculated to check the mortality of G. mellonella or infectivity of different age group of infective stage juveniles. For each treatment three replicates were used.

Freshly hatched infective stage juvenile of EPN isolates viz., S. pakistanese; S. asiaticum; S. feltiae; Steinernema abbasi; Steinernema siamkayai; Heterorhabditis indica and H. bacteriophora had maximum infectivity and showed 100% mortality of G. mellonella larvae.

EPN isolates S. pakistanese; S. asiaticum; S. feltiae; Steinernema abbasi; Steinernema siamkayai; Heterorhabditis indica and H. bacteriophora were tested and two months old storage of infective stage juveniles of above mentioned isolates showed 85, 81, 83, 80, 79, 83 and 79% mortality of G. mellonella larvae, respectively.

EPN isolates S. pakistanese; S. asiaticum; S. feltiae; Steinernema abbasi; Steinernema siamkayai; Heterorhabditis indica and H. bacteriophora were tested and four months old storage of infective stage juvenile of above mentioned isolates showed 73, 69, 70, 67, 63, 70 and 65% mortality of G. mellonella larvae, respectively.

EPN isolates S. pakistanese; S. asiaticum; S. feltiae; Steinernema abbasi; Steinernema siamkayai; Heterorhabditis indica and H. bacteriophora were tested and six months old storage of infective stage juvenile of above mentioned isolates showed 55, 54.5 54, 51, 49, 53 and 48% mortality of G. mellonella larvae, respectively.

EPN isolates S. pakistanese; S. asiaticum; S. feltiae; Steinernema abbasi; Steinernema siamkayai; Heterorhabditis indica and H. bacteriophora were tested and eight months old storage of infective stage juvenile of above mentioned isolates showed 44, 45, 43, 39, 31, 40 and 32% mortality of G. mellonella larvae, respectively.

EPN isolates S. pakistanese; S. asiaticum; S. feltiae; Steinernema abbasi; Steinernema siamkayai; Heterorhabditis indica and H. bacteriophora were tested and ten months old storage of infective stage juvenile of above mentioned isolates showed 30, 29, 28, 23, 19, 29 and 23% mortality of G. mellonella larvae, respectively.

EPN isolates Steinernema pakistanese; S. asiaticum; S. feltiae; S. abbasi; S. siamkayai; Heterorhabditis indica and H. bacteriophora were tested and twelve months old storage of infective stage juvenile of above mentioned isolates showed 14, 14, 14, 11, 09, 13, and 11% mortality of G. mellonella larvae respectively.

It could be concluded that EPN can be successfully cultured on a large scale and could be used under field condition to provide protection to the crops from insects.

### REFERENCES

Akhurst, R. J. & Boemare, N. E. (1988). A numerical taxonomic study of the genus Xenorhabdus (Enterobacteriaceae) and proposed elevation of the subspecies of X. nematophilus to species. J. Gen. Microbiol., 134: 1835-1845.

Anis, M., Shahina, F., Reid, A. P. & Janet Rowe (2002). Steinernema asiaticum sp.n. (Rhabditida: Steinernematidae) from Pakistan. Int. J. Nematol., 12: 220-231.

Bedding, R. A. (1994). Methods for the storage of Entomopathogenic nematodes. WO Patent No. 1994005150.

Bedding, R. A. (1991). Storage of Entomopathogenic nematodes. U.S. Pat. No. 5,042,427.

Bedding, R. A. (1984). Large-scale production, storage and transport of the insect parasitic nematodes Neoaplectana spp. and Heterorhabditis spp. Ann. Appl. Biol., 104: 117-120.

Bedding, R. A. & Akhurst, R. J. (1975). A simple technique for the detection of insect parasitic rhabditid nematodes in soil. Nematologica, 21: 109-110.

Boemare, N. E., Akhurst, R. J. & Mourant, R. G. (1993). DNA relatedness between Xenorhabdus spp. (Enterobacteriaceae), symbiotic bacteria of entomopathogenic nematodes and a proposal to transfer Xenorhabdus luminescens to a new genus, Photorhabdus gen. nov. Int. J. Sys. Bacteriol., 43:249-255.

Chitwood, B. G. & Chitwood, M. B. (1937). An Introduction to Nematology. Monumental Printing Co., Baltimore, Md.

Elawad, A. S., Ahmad, W. & Reid, A. P. (1997). Steinernema abbasi sp. n. (Nematoda: Steinernematidae) from the Sultanate of Oman. Fun. App. Nematol., 20: 435-442.

Filipjev, I. N. (1934). Eine new art der gattung Neoplectana Steiner nebst Bemmerkungen uber die systematishe sellung der letzteren. Magasin de parasitologie de I'Institute Zoologique des Sciences de I'USSR. IV. 1934. 229-240.

Griffin, C. T. (1996). Effect of prior storage conditions on the infectivity of Heterorhabditis spp. (Nematoda: Heterorhabditidae). Fun. App. Nematol., 19: 95-102.

Nguyen, K. B. and G. C. Smart, Jr. (1996). Identification of entomopathogenic nematodes in the Steinernematidae and Heterorhabditidae (Nematoda: Rhabditida). J. Nematol., 28, 286-300.

Poinar, G. O. (1976). Description and biology of a new insect parasitic rhabditoid, Heterorhabditis bacteriophora n. gen., n. sp. (Rhabditida: Heterorhabditidae n. fam.) Nematologica, 21: 463-470.

Poinar, G. 0. Jr. (1975). Entomogenous nematodes. A manual and host list of insect-nematode associations. Leiden, The Netharlands: E. J. Brill, 317 pp.

Poinar, G. O., Karunakar, G. K. & David, H. (1992). Heterorhabditis indicus n. sp. (Rhabditida: Nematoda) from India: Separation of Heterorhabditis spp. by infective juveniles. Fun. App. Nematol., 15: 467-472.

Shahina, F., Anis, M., Reid, A. P., Rowe, J. & Maqbool, M. A. (2001). Steinernema pakistanense sp.n. (Rhabditida: Steinernematidae) from Pakistan. Int. J. Nematol., 11: 124-133.

Shahina, F. & Maqbool, M. A. (1996). Isolation of entomopathogenic nematodes (Heterorhabditidae and Steinernematidae) from Pakistan. Pak. J. Nematol., 14: 135-136.

Stock, S. P., Somsook, V. & Ried, A. P. (1998). Steinernema siamkayai n. sp. (Rhabditida: Steinernematidae) an entomopathogenic nematode from Thailand. Sys. Parasitol., 41: 105-113.

Tabassum, K. A. & Shahina, F. (2004). In vitro mass rearing of different species of entomopathogenic nematodes in monoxenic solid culture. Pak. J. Nematol., 22: 167-175.

Tachibana, M., Indrasith, L. Suzuki, N. & Asano, I. (1998). Process for producing entomopathogenic nematode preparation and method of storing the same. PCT. JP9603067.

Travassos, L. (1927). Sobre O genera oxysomatium. Bol. Biol. Sao Paolo, 5: 20-21.

Thomas, G. M. & Poinar, G. O. Jr. (1979). Xenorhabdus gen. nov., a genus of entomopathogenic nematophilic bacteria of the family Enterobacteriaceae Int. J. Sys. Bacteriol., 29: 352-360.

White, G. F. (1927). A method for obtaining infective nematode larvae from cultures. Science, 66: 302-303.

Yukawa, Pitt, Janice, M., Takao (1988). Nematode storage and transport. U.S. Pat. No. 4,765,275.

What is claimed is:

1. A method for storing infective stage juveniles of entomopathogenic nematodes belonging to the genera of Steinernema and Heterorhabditis comprising:
a. Dispersing nematodes in a 0.1-0.5% mixture of surfactant Trirton X-100 in distilled water
b. Contacting the above dispersion of nematodes on to the surface of an autoclaved polyether polyurethane foam such that the quantity of nematodes suspension contacted is 5-15 times the weight of said foam
c. Storing said polyurethane foam in a growth chamber at 10-15 C

2. A method for storing infective stage juveniles of entomopathogenic nematodes belonging to the genera of Steinernema and Heterorhabditis comprising:
a. Dispersing nematodes in a 0.1-0.5% mixture of the antimicrobial agent formalin in distilled water
b. Contacting the above dispersion of nematodes on to the surface of an autoclaved polyether polyurethane foam such that the quantity of nematodes suspension is 5-15 times the weight of said foam
c. Storing said polyurethane foam in a growth chamber at 10-15 C

3. As claimed in claims 1 and 2 wherein said infective juveniles of entomopahtogenic nematodes are selected from a group comprising of Steinernema pakistanensis, Steinernema asiaticum, Steinernema feltiae, Steinernema abbasi, Steinernema. siamkayai, Heterorhabditis indica, and Heterorhabditis bacteriophora.

4. As claimed in claims 1 and 2 wherein said infective juveniles of entomopahtogenic nematodes retain their infectiveness of at least 50% for six-month period of storage.
